(19) United States
(12) Patent Application Publication
(10) Pub. No.: US 2024/0009099 A1
(43) Pub. Date: Jan. 11, 2024

(54) ANHYDROUS SOLID SHAMPOO BASED ON SURFACTANTS AND ON A FATTY ACID SALT, ITS METHOD OF PREPARATION AND A KIT

(71) Applicant: L'OREAL, Paris (FR)

(72) Inventors: Frederik PINAY, Saint Ouen (FR); Mélanie OTT, Saint Ouen (FR); Maxime ROYER, Saint Ouen (FR)

(73) Assignee: L'OREAL, Paris (FR)

(21) Appl. No.: 18/255,830

(22) PCT Filed: Dec. 3, 2021
(86) PCT No.: PCT/EP2021/084251

§ 371 Date: Jun. 2, 2023

(30) Foreign Application Priority Data
Dec. 3, 2020 (FR) 2012601

(51) Int. Cl. A61K 8/46; A61Q 5/02; A61Q 5/12; A61K 8/02; A61K 8/44; A61K 8/36; A61K 8/73

(52) U.S. Cl. CPC A61K 8/463; A61Q 5/02; A61Q 5/12; A61K 8/0216; A61K 8/442; A61K 8/361; A61K 8/737; A61K 2800/31; A61K 2800/596; A61K 2800/5426; A61K 2800/87

(57) ABSTRACT

The present invention relates to an anhydrous solid composition comprising one or more anionic surfactants, one or more amphoteric or zwitterionic surfactants and one or more C₈-C₃₂ fatty acid salts. The invention also relates to a method for the preparation of an aqueous composition for washing, and optionally in addition conditioning, keratin fibres starting from the anhydrous solid composition according to the invention, to the use of an anhydrous solid composition according to the invention for washing, and optionally in addition conditioning, keratin fibres, and also to a kit for the preparation of the aqueous composition for washing, and optionally in addition conditioning, keratin fibres.

The present invention relates to an anhydrous solid composition comprising one or more anionic surfactants, one or more amphoteric or zwitterionic surfactants and one or more fatty acid salts.

The invention also relates to a method for the preparation of an aqueous composition for washing, and optionally in addition conditioning, keratin fibres, in particular the hair, starting from the anhydrous solid composition according to the invention, to the use of an anhydrous solid composition according to the invention for washing, and optionally in addition conditioning, keratin fibres, and also to a kit for the preparation of the aqueous composition for washing, and optionally in addition conditioning, keratin fibres.

In the field of hair hygiene, products for washing and/or caring for keratin fibres are predominantly, not to say completely, packaged in plastic-based packagings, for example in the nonbiodegradable plastic bottle or plastic sachet form.

To date, the field of cosmetics is dependent on these plastic-based packagings, which are not much recycled or are difficult to recycle, and solutions for being liberated from them are rare.

Packagings based on paper or on biodegradable materials for aqueous compositions for washing and/or caring for keratin fibres are not entirely satisfactory. This is because this type of packaging can disintegrate in the presence of water, which does not make possible the storage of these compositions or make it possible to retain good stability over time.

For all that, for the sake of protecting the environment, consumers are increasingly looking at the plastic footprint of their purchases and are demanding cosmetic products which are packaged without plastics or materials resulting from petrochemicals, and in smaller packagings.

Furthermore, still for the sake of protecting the environment, consumers are also concerned about the carbon footprint of their purchases, that is to say about the amount of carbon dioxide generated during the production of the cosmetic product up to its use, going through the distribution of the product.

In the past, solid shampoos in the form of cakes or bars could be marketed. However, these shampoos in the form of cakes or bars were not entirely satisfactory, in particular as regards the application of the solid shampoo to the hair. This is because it appears that it is more difficult for the user to homogeneously apply to the hair an effective dose of a solid shampoo in the cake or bar form than the same dose with a conventional aqueous shampoo.

There thus exists a real need to develop cosmetic products exhibiting a reduced plastic footprint and a reduced carbon footprint. In particular, there is a need to develop cosmetic compositions capable of being packaged in nonplastic packagings or packagings not resulting from petrochemicals, and in smaller packagings, to limit the generation of carbon dioxide throughout the life of the product, this being achieved while conferring, on keratin fibres, in particular the hair, washing and/or conditioning performance qualities which are expected as regards washing power, suppleness, feel, softness, sheen and disentangling.

In addition, it is also advantageous for these compositions to make it possible to recreate ready-for-use aqueous washing/conditioning compositions for keratin fibres, in particular the hair, and for these ready-for-use aqueous compositions to be able to be recreated easily by the user himself, and this well before its use.

These aims are achieved with the present invention, which has in particular for subject-matter an anhydrous solid composition comprising:
(i) one or more anionic surfactants,
(ii) one or more amphoteric or zwitterionic surfactants, and
(iii) from 0.1% to 15% by weight, with respect to the total weight of the anhydrous solid composition, of one or more C₈-C₃₂ fatty acid salts; and
   the total content of surfactants being greater than or equal to 60% by weight, with respect to the total weight of the anhydrous solid composition.

The anhydrous solid composition according to the invention makes it possible for the user to easily prepare, at home, a ready-for-use aqueous composition for washing, and optionally in addition for conditioning, keratin fibres, such as the hair, for example a shampoo, by mixing the anhydrous solid composition according to the invention and water in a bottle, for example a glass bottle. The ready-for-use aqueous composition thus prepared can be used for several days and even several weeks after it has been prepared.

The anhydrous solid composition according to the invention exhibits the advantage of being able to be packaged in nonplastic packagings, for example in sachets made of paper or of biodegradable or metallic materials, and also to be packaged in smaller packagings than a conventional shampoo.

The anhydrous solid composition according to the invention also exhibits the advantage of having a reduced weight and a reduced volume, in comparison with a conventional aqueous washing composition, for example a shampoo of 250 ml packaged in a polyethylene terephthalate bottle. In other words, for one unit of anhydrous solid composition, equivalent to one unit of washing aqueous composition once reformulated with water or also to 250 ml of conventional shampoo, the weight and the volume of the anhydrous solid composition are lower.

This reduction in weight and in volume per unit of anhydrous solid composition makes it possible to significantly reduce the amount of carbon dioxide generated during the transportation of the compositions according to the invention from the production plant as far as the distribution points, indeed even as far as the homes of the consumers.

This is because the reduction in the volume per unit of composition makes it possible to deliver more of it with one and the same means of transportation (for example a truck or a plane) and thus to reduce the amount of carbon dioxide generated, with respect to each unit of composition.

In the same way, the reduction in the weight per unit of composition according to the invention makes it possible to reduce the total weight of the delivery for one and the same means of transportation (for example a truck or a plane), and thus the amount of fuel energy necessary for the delivery (for example gasoline or kerosene), and thus to reduce the amount of carbon dioxide generated, with respect to each unit of composition.

It has also been found that, once reformulated with water, the aqueous washing, and optionally in addition conditioning, compositions thus obtained exhibit a good washing power and confer suppleness, a good feel, softness and sheen, and facilitate the disentangling of keratin fibres.

Advantageously, once reformulated with water, the anhydrous solid compositions according to the invention result in transparent compositions.

Another subject-matter of the invention is a method for the preparation of an aqueous composition for washing, and optionally in addition for conditioning, keratin fibres, in particular human keratin fibres, such as the hair, comprising at least a step of mixing an anhydrous solid composition according to the invention with water.

Another subject-matter of the invention is the use of an anhydrous solid composition according to the invention for washing, and optionally in addition for conditioning, keratin fibres, in particular human keratin fibres, such as the hair.

The invention also relates to a method for the treatment of keratin fibres, comprising the application, to said keratin fibres, of the anhydrous solid composition according to the invention.

The invention also relates to a kit for the preparation of an aqueous composition for washing, and optionally in addition for conditioning, keratin fibres, in particular human keratin fibres, such as the hair, comprising at least a first compartment comprising an anhydrous solid composition according to the invention and at least a second compartment capable of containing the mixture of the anhydrous solid composition according to the invention with water.

Other subject-matters, characteristics, aspects and advantages of the invention will become even more clearly apparent on reading the description and the example which follow.

In the present description, and unless otherwise indicated:
the expression “at least one” is equivalent to the expression “one or more” and can be replaced therewith;
the expression “of between” is equivalent to the expression “ranging from” and can be replaced therewith, and implies that the limits are included;
within the meaning of the present invention, the expression “greater than” and respectively the expression “less than” are understood to mean an open interval which is strictly greater, respectively strictly less, and thus that the limits are not included;
the term “keratin fibres”, according to the present patent application, more particularly denotes human keratin fibres and more preferentially the hair.

The composition according to the invention is solid at ambient temperature (25° C.) and at atmospheric pressure (1.013×10⁵ Pa).

The composition according to the invention is anhydrous.

The term “anhydrous composition” is understood to mean a composition comprising a content of water of less than 5% by weight, preferably of less than 3% by weight, with respect to the weight of the composition. Preferably, this content of water is less than 1% by weight, better still less than 0.5%, indeed even less than 0.3%, by weight, with respect to the weight of the composition. More particularly, it does not comprise water (0%).

In particular, the anhydrous solid composition does not comprise water added during its preparation, it being possible for the residual water possibly present to originate from the starting materials employed during the preparation.

The anhydrous solid composition according to the invention can be in the form of a powder, a paste, particles (for example spherical particles, such as small beads or granules), a compressed tablet, a stick or a block. Preferably, the composition according to the invention is provided in the form of a powder or particles, and more preferentially in the form of a powder.

The term “powder” is understood to mean a composition in the pulverulent form, which is preferably essentially devoid of dust (or fine particles). In other words, the particle size distribution of the particles is such that the content by weight of the particles which have a size of less than or equal to 50 micrometres (content of fines), preferably of less than or equal to 45 micrometres (content of fines), is advantageously less than or equal to 5% by weight, preferably less than 3% by weight and more particularly less than 1% by weight, with respect to the total weight of the particles (size of the particles evaluated by means of a Retsch AS 200 Digit particle size analyser; oscillation height: 1.25 mm/sieving time: 5 minutes). Advantageously, the size of the powder particles is between 5 μm and 3 mm, preferably between 10 μm and 2 mm, more preferentially between 50 μm and 1 mm, and better still between 60 μm and 600 μm.

The term “paste” is understood to mean a composition exhibiting a viscosity of greater than 5 poises (500 mPa·s) and preferably of greater than 10 poises (1000 mPa·s), measured at 25° C. and at a shear rate of 1 s⁻¹, it being possible for this viscosity to be determined using a cone-plate rheometer.

The term “particles” is understood to mean small fractionated objects formed of solid particles aggregated together, of variable shapes and sizes. They can be regular or irregular in shape. They can in particular be of spherical shape (such as granules, granular material, beads), square shape, rectangular shape or elongated shape such as rods. Spherical particles are very particularly preferred. The size of the particles can advantageously be, in its greatest dimension, between 5 μm and 5 mm, preferably between 10 μm and 2 mm, more preferentially between 50 μm and 1000 μm and better still between 60 μm and 600 μm.

When the anhydrous solid composition according to the invention is not provided in the form of a powder or particles, the composition preferably exhibits a force of penetration at 25° C. and 1 atm (1.013×10⁵ Pa) of greater than or equal to 200 g, in particular of greater than or equal to 300 g, indeed even 400 g, better still 500 g. The force of penetration is determined by penetrometry. The texture analysis measurements are carried out at 25° C. using a Stable Micro Systems TA.XT Plus texture analyser. The penetrometry experiments are carried out with a metal rod provided with a screw tip, said tip being a P/2N needle of 2 mm for the top part, which tip is connected to the measuring head. The piston is driven into the sample at a constant rate of 1 mm/s, over a height of 5 mm. The force exerted on the piston is recorded and the mean value of the force is calculated.

The anhydrous solid composition according to the invention can be provided in the form of a compressed anhydrous solid composition, in particular compressed using a manual or mechanical press.

The density of the anhydrous solid composition according to the present invention is preferably of between 0.1 and 1, more preferentially between 0.2 and 0.8, and better still between 0.3 and 0.6.

By way of example, the density can be measured according to the following method: A predetermined amount (weight, w) of powder is placed in a 250 ml graduated measuring cylinder. The powder contained in the measuring cylinder will subsequently undergo 2500 compressions in order to obtain the volume (v) occupied by the powder. The volume (v) thus obtained is read on the measuring cylinder and the density (d) is subsequently determined according to the formula d=w/v.

The Anionic Surfactants

The anhydrous solid composition according to the present invention comprises one or more anionic surfactants.

The term “anionic surfactant” is understood to mean a surfactant comprising, as ionic or ionizable groups, only anionic groups.

In the present description, an entity is described as being “anionic” when it possesses at least one permanent negative charge or when it can be ionized to give a negatively charged entity, under the conditions of use of the composition of the invention (medium or pH, for example), and not comprising a cationic charge.

The anionic surfactants can be chosen from sulfate, sulfonate and carboxylic (or carboxylate) surfactants. Very obviously, a mixture of these surfactants can be employed.

It is understood, in the present description, that:
the carboxylate anionic surfactants comprise at least one carboxyl or carboxylate (—COOH or —COO⁻) functional group and can optionally additionally comprise one or more sulfate and/or sulfonate functional groups;
the sulfonate anionic surfactants comprise at least one sulfonate (—SO₃H or —SO₃⁻) functional group and can optionally additionally comprise one or more sulfate functional groups, but do not comprise a carboxylate functional group; and
the sulfate anionic surfactants comprise at least one sulfate functional group but do not comprise a carboxylate or sulfonate functional group.

The carboxylate anionic surfactants capable of being used thus comprise at least one carboxyl or carboxylate (—COOH or —COO⁻) functional group.

The carboxylate anionic surfactants can be chosen from the following compounds: acylglycinates, acyllactylates, acylsarcosinates, acylglutamates; alkyl-D-galactosideuronic acids, alkyl ether carboxylic acids, alkyl(C₆-C₃₀ aryl) ether carboxylic acids, alkylamido ether carboxylic acids; and also the salts of these compounds; and their mixtures;
the alkyl and/or acyl groups of these compounds comprising from 6 to 30 carbon atoms, in particular from 12 to 28, better still from 14 to 24, indeed even from 16 to 22, carbon atoms; the aryl group preferably denoting a phenyl or benzyl group;
it being possible for these compounds to be polyoxyalkylenated, in particular polyoxyethylenated, and then preferably comprising from 1 to 50 ethylene oxide units, better still from 2 to 10 ethylene oxide units.

Use may also be made of C₆-C₂₄ alkyl monoesters of polyglycoside-polycarboxylic acids, such as C₆-C₂₄ alkyl polyglycoside-citrates, C₆-C₂₄ alkyl polyglycoside-tartrates and C₆-C₂₄ alkyl polyglycoside-sulfosuccinates, and their salts.

Preferentially, the carboxylate anionic surfactants are chosen from, alone or as a mixture:
acylglutamates, in particular C₆-C₂₄, indeed even C₁₂-C₂₀, acylglutamates, such as stearoylglutamates, and especially disodium stearoylglutamate;
acylsarcosinates, in particular C₆-C₂₄, indeed even C₁₂-C₂₀, acylsarcosinates, such as palmitoylsarcosinates, and especially sodium palmitoylsarcosinate;
acyllactylates, in particular C₁₂-C₂₈, indeed even C₁₄-C₂₄, acyllactylates, such as behenoyllactylates, and especially sodium behenoyllactylate;
C₆-C₂₄, in particular C₁₂-C₂₀, acylglycinates;
(C₆-C₂₄)alkyl ether carboxylates, and in particular (C₁₂-C₂₀)alkyl ether carboxylates;
polyoxyalkylenated (C₆-C₂₄)alkyl(amido) ether carboxylic acids, in particular those comprising from 2 to 50 ethylene oxide groups;
in particular in the form of alkali metal or alkaline earth metal, ammonium or aminoalcohol salts.

Mention may very particularly be made, among the above carboxylic surfactants, of surfactants of sarcosinate type, in particular chosen from (C₆-C₃₀)acylsarcosinates of following formula (I):

R—C(O)—N(CH₃)—CH₂—C(O)—OX (I)
with:
   X denoting a hydrogen atom, an ammonium ion, an ion resulting from an alkali metal or alkaline earth metal or an ion resulting from an organic amine, preferably a hydrogen atom, and
   R denoting a linear or branched alkyl group of 5 to 29 carbon atoms.
R preferably denotes a linear or branched alkyl group of 8 to 24 carbon atoms, preferably of 12 to 20 carbon atoms.

Mention may be made, among the (C₆-C₃₀)acylsarcosinates of formula (I) which can be used in the present composition, of palmitoylsarcosinates, stearoylsarcosinates, myristoylsarcosinates, lauroylsarcosinates and cocoylsarcosinates, in acid form or in salified form.

The anionic surfactant(s) of sarcosinate type are advantageously chosen from sodium lauroylsarcosinate, stearoylsarcosine, myristoylsarcosine and their mixtures, preferably from stearoylsarcosine, myristoylsarcosine and their mixtures.

Mention may also be made, among the carboxylic surfactants above, of polyoxyalkylenated alkyl(amido) ether carboxylic acids and their salts, in particular those comprising from 2 to 50 alkylene oxide groups, in particular ethylene oxide groups, such as the compounds provided by Kao under the Akypo names.

The polyoxyalkylenated alkyl(amido) ether carboxylic acids capable of being used are preferably chosen from those of formula (II):

R₁—(OC₂H₄)ₙ—OCH₂COOA (II)
in which:
   R1 represents a linear or branched C₆-C₂₄ alkyl or alkenyl radical, a (C₈-C₉)alkylphenyl radical or an R₂CONH—CH₂—CH₂— radical with R2 denoting a linear or branched C₉-C₂₁ alkyl or alkenyl radical;
preferably, R1 is a C₈-C₂₀, preferably C₈-C₁₈, alkyl radical and aryl preferably denotes phenyl,
   n is an integer or decimal number (mean value) varying from 2 to 24, preferably from 2 to 10,
   A denotes H, ammonium, Na, K, Li, Mg or a monoethanolamine or triethanolamine residue.

Use may also be made of mixtures of compounds of formula (II), in particular of mixtures of compounds having different R1 groups.

The polyoxyalkylenated alkyl(amido) ether carboxylic acids which are particularly preferred are those of formula (II) in which:
R1 denotes a C₁₂-C₁₄ alkyl, cocoyl, oleyl, nonylphenyl or octylphenyl radical,
A denotes a hydrogen or sodium atom, and
n varies from 2 to 20, preferably from 2 to 10.

More preferentially still, use is made of compounds of formula (II) in which R1 denotes a C₁₂ alkyl radical, A denotes a hydrogen or sodium atom and n varies from 2 to 10.

The sulfonate anionic surfactants capable of being used comprise at least one sulfonate (—SO₃H or —SO₃⁻) functional group.

The sulfonate anionic surfactants can be chosen from the following compounds: alkylsulfonates, alkylamidesulfonates, alkylarylsulfonates, α-olefinsulfonates, paraffinsulfonates, alkyl sulfosuccinates, alkyl ether sulfosuccinates, alkylamide sulfosuccinates, alkyl sulfoacetates, N-acyl taurates, acyl isethionates, alkyl sulfolaurates, and also the salts of these compounds;
the alkyl groups of these compounds comprising from 6 to 30 carbon atoms, in particular from 12 to 28, better still from 14 to 24, indeed even from 16 to 22, carbon atoms; the aryl group preferably denoting a phenyl or benzyl group;
it being possible for these compounds to be polyoxyalkylenated, in particular polyoxyethylenated, and then preferably comprising from 1 to 50 ethylene oxide units, better still from 2 to 10 ethylene oxide units.

Preferentially, the sulfonate anionic surfactants are chosen from, alone or as a mixture:
C₆-C₂₄, in particular C₁₂-C₂₀, alkyl sulfosuccinates, in particular lauryl sulfosuccinates;
C₆-C₂₄, in particular C₁₂-C₂₀, alkyl ether sulfosuccinates;
N—(C₆-C₂₄)acyl taurates, in particular N—(C₁₂-C₂₀)acyl taurates;
(C₆-C₂₄)acyl isethionates, preferably (C₁₂-C₁₈)acyl isethionates;
in particular in the form of alkali metal or alkaline earth metal, ammonium or aminoalcohol salts.

Preferably, the anionic surfactant(s) of sulfonate type are chosen from N—(C₆-C₂₄)acyl taurates, in particular N—(C₁₂-C₂₀)acyl taurates, and especially N-acyl N-methyl taurates, C₆-C₂₄ acyl isethionates, in particular C₁₂-C₁₈ acyl isethionates, and also their salts and their mixtures.

More preferentially, the anionic surfactant(s) of sulfonate type are chosen from C₆-C₂₄ acyl isethionates, in particular C₁₂-C₁₈ acyl isethionates, and also their salts and their mixtures.

The sulfate anionic surfactants capable of being used comprise at least one sulfate (—OSO₃H or —OSO₃⁻) functional group.

The sulfate anionic surfactants can be chosen from the following compounds: alkyl sulfates, alkyl ether sulfates, alkylamido ether sulfates, alkylaryl polyether sulfates, monoglyceride sulfates; and also the salts of these compounds;
the alkyl groups of these compounds comprising from 6 to 30 carbon atoms, in particular from 8 to 28, better still from 10 to 24, indeed even from 12 to 22, carbon atoms; the aryl group preferably denoting a phenyl or benzyl group;
it being possible for these compounds to be (poly)oxyalkylenated, in particular (poly)oxyethylenated, and then preferably comprising from 1 to 50 ethylene oxide units, better still from 1 to 10 ethylene oxide units.

Preferentially, the sulfate anionic surfactants are chosen from, alone or as a mixture:
alkyl sulfates, in particular C₁₀-C₂₄, indeed even C₁₂-C₂₂, alkyl sulfates;
alkyl ether sulfates, in particular C₁₀-C₂₄, indeed even C₁₂-C₂₂, alkyl ether sulfates, preferably comprising from 1 to 20 ethylene oxide units;
in particular in the form of alkali metal or alkaline earth metal, ammonium or aminoalcohol salts.

When the anionic surfactant is in the salt form, said salt can be chosen from alkali metal salts, such as the sodium or potassium salt, ammonium salts, amine salts and in particular aminoalcohol salts, and alkaline earth metal salts, such as the magnesium salt.

Mention may be made, as examples of aminoalcohol salts, of mono-, di- and triethanolamine salts, mono-, di- or triisopropanolamine salts or 2-amino-2-methyl-1-propanol, 2-amino-2-methyl-1,3-propanediol and tris(hydroxymethyl)aminomethane salts.

The alkali metal or alkaline earth metal salts and in particular the sodium or magnesium salts are preferably used.

Advantageously, the anionic surfactant(s) are chosen from sulfate anionic surfactants.

More preferentially, the anionic surfactant(s) are chosen from alkyl sulfates, alkyl ether sulfates, alkylamido ether sulfates, alkylaryl polyether sulfates, monoglyceride sulfates, mixtures thereof and also the salts of these compounds;
the alkyl groups of these compounds comprising from 6 to 30 carbon atoms, in particular from 8 to 28, better still from 10 to 24, indeed even from 12 to 22, carbon atoms; the aryl group preferably denoting a phenyl or benzyl group;
it being possible for these compounds to be (poly)oxyalkylenated, in particular (poly)oxyethylenated, and then preferably comprising from 1 to 50 ethylene oxide units, better still from 1 to 10 ethylene oxide units.

More preferentially still, the anionic surfactant(s) are chosen from:
C₆-C₃₀, better still C₈-C₂₄, even better still C₁₀-C₂₄, indeed even C₁₂-C₂₂, alkyl sulfates;
C₆-C₂₄, better still C₁₀-C₂₄, indeed even C₁₂-C₂₂, alkyl ether sulfates; preferably comprising from 1 to 20 ethylene oxide units;
and their mixtures;
in particular in the form of alkali metal or alkaline earth metal, ammonium or aminoalcohol salts.

More preferentially still, the anionic surfactant(s) are chosen from C₆-C₃₀, better still C₈-C₂₄, even better still C₁₀-C₂₄, indeed even C₁₂-C₂₂, alkyl sulfates.

Preferably, the anhydrous solid composition comprises at least two anionic surfactants, preferably at least two sulfate anionic surfactants, more preferentially at least two C₆-C₃₀, better still C₈-C₂₄, even better still C₁₀-C₂₄, indeed even C₁₂-C₂₂, alkyl sulfates.

Preferably, the total content of anionic surfactant(s) present in the anhydrous solid composition ranges from 55% to 90% by weight, more preferentially from 56% to 85% by weight, more preferentially still from 57% to 80% by weight, better still from 58% to 75% by weight, even better still from 59% to 75% by weight and still better from 60% to 75% by weight, with respect to the total weight of the anhydrous solid composition.

Preferably, the total content of sulfate anionic surfactant(s) present in the anhydrous solid composition ranges from 55% to 90% by weight, more preferentially from 56% to 85% by weight, more preferentially still from 57% to 80% by weight, better still from 58% to 75% by weight, even better still from 59% to 75% by weight and still better from 60% to 75% by weight, with respect to the total weight of the anhydrous solid composition.

Preferably, the total content of C₆-C₃₀ alkyl sulfates present in the anhydrous solid composition ranges from 55% to 90% by weight, more preferentially from 56% to 85% by weight, more preferentially still from 57% to 80% by weight, better still from 58% to 75% by weight, even better still from 59% to 75% by weight and still better from 60% to 75% by weight, with respect to the total weight of the anhydrous solid composition.

The Amphoteric or Zwitterionic Surfactants

The anhydrous solid composition according to the present invention comprises one or more amphoteric or zwitterionic surfactants.

In particular, the amphoteric or zwitterionic surfactant(s), which are preferably non-silicone, used in the anhydrous solid composition according to the present invention can in particular be derivatives of optionally quaternized aliphatic secondary or tertiary amines, in which derivatives the aliphatic group is a linear or branched chain comprising from 8 to 22 carbon atoms, said amine derivatives containing at least one anionic group, such as, for example, a carboxylate, sulfonate, sulfate, phosphate or phosphonate group.

Mention may in particular be made of (C₈-C₂₀)alkyl betaines, (C₈-C₂₀)alkyl sulfobetaines, (C₈-C₂₀)alkylamido(C₁-C₆)alkyl betaines, (C₈-C₂₀)alkylamido(C₁-C₆)alkyl sulfobetaines, and their mixtures.

Mention may be made, among the derivatives of optionally quaternized aliphatic secondary or tertiary amines which can be used, as defined above, of the compounds with the following respective structures (III) and (IV):

Rₐ—CONHCH₂CH₂—N⁺(R_{b})(R_{c})—CH₂COO⁻·M⁺·X⁻ (III)
in which formula (III):
   Rₐ represents a C₁₀ to C₃₀ alkyl or alkenyl group derived from an acid RₐCOOH preferably present in hydrolysed coconut oil; preferably, Rₐ represents a heptyl, nonyl or undecyl group;
   R_{b} represents a β-hydroxyethyl group;
   R_{c} represents a carboxymethyl group;
   M⁺ represents a cationic counterion resulting from an alkali or alkaline earth metal, such as sodium, an ammonium ion or an ion resulting from an organic amine; and
   X⁻ represents an organic or inorganic anionic counterion, such as that chosen from halides, acetates, phosphates, nitrates, (C₁-C₄)alkyl sulfates or (C₁-C₄)alkyl- or (C₁-C₄)alkylarylsulfonates, in particular methyl sulfate and ethyl sulfate; or alternatively M⁺ and X⁻ are absent;

Rₐ′—CONHCH₂CH₂—N(B)(B′) (IV)
in which formula (IV):
   B represents the —CH₂CH₂OX′ group;
   B′ represents the —(CH₂)_{z}Y′ group, with z=1 or 2;
   X′ represents the —CH₂COOH, —CH₂—COOZ′, —CH₂CH₂COOH or CH₂CH₂—COOZ′ group, or a hydrogen atom;
   Y′ represents the —COOH, —COOZ′ or —CH₂CH(OH)SO₃H group or the CH₂CH(OH)SO₃—Z′ group;
   Z′ represents a cationic counterion resulting from an alkali or alkaline earth metal, such as sodium, an ammonium ion or an ion resulting from an organic amine;
   Rₐ′ represents a C₁₀ to C₃₀ alkyl or alkenyl group of an acid Rₐ′—COOH preferably present in hydrolysed linseed oil or coconut oil, preferably Rₐ′ an alkyl group, in particular a C₁₇ alkyl group and its iso form, an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

Mention may be made, by way of example, of the cocoamphodiacetate sold by Rhodia under the trade name Miranol® C2M Concentrate.

Use may also be made of the compounds of formula (V):

Rₐ″—NHCH(Y″)—(CH₂)ₙCONH(CH₂)_{n′}—N(R_{d})(Rₑ) (V)
in which formula (V):
   Y″ represents the —COOH, —COOZ″ or —CH₂—CH(OH)SO₃H group or the CH₂CH(OH)SO₃—Z″ group;
   R_{d} and Rₑ, independently of each other, represent a C₁ to C₄ alkyl or hydroxyalkyl radical;
   Z″ represents a cationic counterion resulting from an alkali or alkaline earth metal, such as sodium, an ammonium ion or an ion resulting from an organic amine;
   Rₐ″ represents a C₁₀ to C₃₀ alkyl or alkenyl group of an acid Rₐ″—COOH preferably present in hydrolysed linseed oil or coconut oil; and
   n and n′ denote, independently of each other, an integer ranging from 1 to 3.

Mention may be made, among the compounds of formula (V), of the compound classified in the CTFA dictionary under the name sodium diethylaminopropyl cocoaspartamide and sold by Chimex under the name Chimexane HB.

These compounds can be used alone or as mixtures.

Use is advantageously made, among the abovementioned amphoteric or zwitterionic surfactants, of (C₈-C₂₀)alkyl betaines, such as cocoyl betaine, and (C₈-C₂₀)alkylamido(C₃-C₈)alkylbetaines, such as cocamidopropyl betaine, alkyl(C₈-C₂₀)amphoacetates, alkyl(C₈-C₂₀)amphodiacetates and their mixtures.

More preferentially, the amphoteric or zwitterionic surfactant(s) are chosen from (C₈-C₂₀)alkyl betaines, (C₈-C₂₀)alkylamido(C₃-C₈)alkyl betaines and their mixtures; more preferentially still from coco betaine, cocamidopropyl betaine and their mixtures.

Better still, the amphoteric or zwitterionic surfactant(s) are chosen from (C₈-C₂₀)alkylamido(C₃-C₈)alkyl betaines and very particularly cocamidopropyl betaine.

Preferably, the total content of amphoteric or zwitterionic surfactant(s) present in the anhydrous solid composition ranges from 5% to 40% by weight, more preferentially from 6% to 35% by weight, more preferentially still from 7% to 30% by weight, better still from 8% to 20% by weight, with respect to the total weight of the anhydrous solid composition.

Preferably, the total content of (C₈-C₂₀)alkyl betaine(s) and (C₈-C₂₀)alkylamido(C₃-C₈)alkyl betaine(s) present in the anhydrous solid composition ranges from 5% to 40% by weight, more preferentially from 6% to 35% by weight, more preferentially still from 7% to 30% by weight, better still from 8% to 20% by weight, with respect to the total weight of the anhydrous solid composition.

Preferably, the total content of (C₈-C₂₀)alkylamido(C₃-C₈)alkyl betaine(s), such as cocamidopropyl betaine, present in the anhydrous solid composition ranges from 5% to 40% by weight, more preferentially from 6% to 35% by weight, more preferentially still from 7% to 30% by weight, better still from 8% to 20% by weight, with respect to the total weight of the anhydrous solid composition.

Advantageously, the total content of surfactants present in the anhydrous solid composition according to the invention ranges from 60% to 95% by weight, more preferentially from 65% to 90% by weight, more preferentially still from 70% to 85% by weight, with respect to the total weight of the anhydrous solid composition.

Preferably, the total content of anionic surfactant(s) and of amphoteric or zwitterionic surfactant(s) present in the anhydrous solid composition ranges from 60% to 95% by weight, more preferentially from 65% to 90% by weight, more preferentially still from 70% to 85% by weight, with respect to the total weight of the anhydrous solid composition.

Preferably, the total content of sulfate anionic surfactant(s) and of amphoteric or zwitterionic surfactant(s) present in the anhydrous solid composition ranges from 60% to 95% by weight, more preferentially from 65% to 90% by weight, more preferentially still from 70% to 85% by weight, with respect to the total weight of the anhydrous solid composition.

Preferably, the total content of C₆-C₃₀ alkyl sulfates and of (C₈-C₂₀)alkyl betaine(s) and (C₈-C₂₀)alkylamido(C₃-C₈)alkyl betaine(s) present in the anhydrous solid composition ranges from 60% to 95% by weight, more preferentially from 65% to 90% by weight, more preferentially still from 70% to 85% by weight, with respect to the total weight of the anhydrous solid composition.

The C₈-C₃₂ Fatty Acid Salts

The anhydrous solid composition according to the present invention additionally comprises from 0.1% to 15% by weight, with respect to the total weight of the anhydrous solid composition, of one or more C₈-C₃₂ fatty acid salts.

It is understood, within the meaning of the present invention, that said C₈-C₃₂ fatty acid salts are different from the anionic surfactant(s) (i) present in the composition according to the invention.

The fatty acid salts which can be used according to the present invention advantageously comprise from 10 to 20 carbon atoms, more preferentially from 12 to 18 carbon atoms.

They are more particularly salts of fatty acids chosen from caprylic acid, capric acid, lauric acid, myristic acid, palmitic acid, stearic acid, oleic acid and their mixtures, such as cocoate salts.

The fatty acid salts can be chosen from alkali metal, alkaline earth metal or amine salts. The salt can be chosen from sodium, potassium, calcium, magnesium, ammonium, diethanolamine, triethanolamine or triisopropanolamine salts.

Mention may be made, as examples of C₈-C₃₂ fatty acid salts, of:
sodium or potassium caprate,
sodium or potassium caprylate,
sodium or potassium or magnesium or calcium or ammonium or triethanolamine laurate,
sodium or potassium or magnesium or calcium or diethanolamine or triethanolamine or triisopropanolamine myristate,
sodium or potassium or magnesium or triethanolamine palmitate,
sodium or potassium or magnesium or triethanolamine cocoate,
sodium or potassium or magnesium or calcium or ammonium or diethanolamine or triethanolamine stearate,
sodium or potassium or ammonium oleate,
sodium arachidate,
sodium or potassium or calcium behenate.

Advantageously, use is made of a, preferably saturated, monocarboxylic acid salt having from 8 to 32 carbon atoms, more preferentially from 10 to 20 carbon atoms, such as those described above.

Preferably, the fatty acid salt(s) comprising from 8 to 32 carbon atoms are chosen from stearic acid salts, more preferentially from alkali metal and alkaline earth metal salts of stearic acid, and their mixtures.

More preferably, the C₈-C₃₂ fatty acid salt(s) are chosen from C₁₀-C₂₀ fatty acid salts, more preferentially still from C₁₂-C₁₈ fatty acid salts, better still from alkali metal and alkaline earth metal salts of stearic acid, and their mixtures, and better still the C₈-C₃₂ fatty acid salt is magnesium stearate.

Preferably, the total content of C₈-C₃₂ fatty acid salt(s) ranges from 0.5% to 15% by weight, more preferentially from 0.5% to 10% by weight, more preferentially still from 1% to 8% by weight, with respect to the total weight of the anhydrous solid composition.

Preferably, the total content of C₁₀-C₂₀ fatty acid salt(s) ranges from 0.5% to 15% by weight, more preferentially from 0.5% to 10% by weight, more preferentially still from 1% to 8% by weight, with respect to the total weight of the anhydrous solid composition.

Preferably, the total content of C₁₂-C₁₈ fatty acid salt(s) ranges from 0.5% to 15% by weight, more preferentially from 0.5% to 10% by weight, more preferentially still from 1% to 8% by weight, with respect to the total weight of the anhydrous solid composition.

Preferably, the total content of magnesium stearate ranges from 0.5% to 15% by weight, more preferentially from 0.5% to 10% by weight, more preferentially still from 1% to 8% by weight, with respect to the total weight of the anhydrous solid composition.

Cationic Polymers

Preferably, the anhydrous solid composition according to the present invention further comprises one or more cationic polymers.

Within the meaning of the present invention, the expression “cationic polymer” denotes any non-silicone (not comprising a silicon atom) polymer containing cationic groups and/or groups which can be ionized to give cationic groups and not containing anionic groups and/or groups which can be ionized to give anionic groups.

The cationic polymers are not silicone-based (do not comprise a Si—O unit).

The cationic polymers may or may not be associative.

The cationic polymers capable of being used preferably have a weight-average molar mass (Mw) of between 500 and 5×10⁶ approximately, preferably of between 10³ and 3×10⁶ approximately.

Mention may more particularly be made, among the cationic polymers, of:

(1) homopolymers or copolymers derived from acrylic or methacrylic esters or amides and comprising at least one of the units of following formulae:

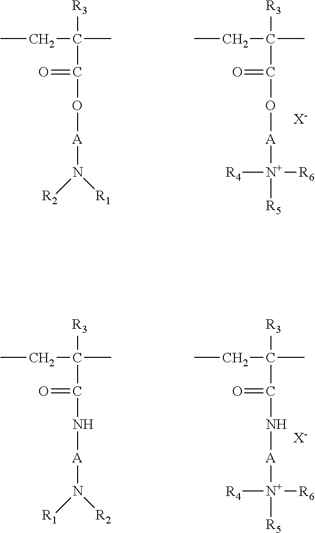

in which formulae:
   R₃, which are identical or different, denote a hydrogen atom or a CH₃ radical;
   A, which are identical or different, represent a linear or branched divalent alkyl group of 1 to 6 carbon atoms, preferably 2 or 3 carbon atoms, or a hydroxyalkyl group of 1 to 4 carbon atoms;
   R₄, R₅ and R₆, which are identical or different, represent an alkyl group having from 1 to 18 carbon atoms or a benzyl radical, preferably an alkyl group having from 1 to 6 carbon atoms;
   R₁ and R₂, which are identical or different, represent a hydrogen atom or an alkyl group having from 1 to 6 carbon atoms, preferably methyl or ethyl; and
   X denotes an anion derived from an inorganic or organic acid, such as a methosulfate anion or a halide, such as chloride or bromide.

The copolymers of the family (1) can additionally contain one or more units deriving from comonomers which can be chosen from the family of the acrylamides, methacrylamides, diacetone acrylamides, acrylamides and methacrylamides substituted on the nitrogen by lower (C₁-C₄) alkyls, acrylic acids or methacrylic acids or their esters, vinyllactams, such as vinylpyrrolidone or vinylcaprolactam, or vinyl esters.

Mention may be made, among these copolymers of the family (1), of:
copolymers of acrylamide and of dimethylaminoethyl methacrylate quaternized with dimethyl sulfate or with a methyl halide, such as that sold under the name Hercofloc by Hercules,
copolymers of acrylamide and of methacryloyloxyethyltrimethylammonium chloride, such as those sold under the name Bina Quat P 100 by Ciba Geigy,
the copolymer of acrylamide and of methacryloyloxyethyltrimethylammonium methosulfate, such as that sold under the name Reten by Hercules,
quaternized or non-quaternized vinylpyrrolidone/dialkylaminoalkyl acrylate or methacrylate copolymers, such as the products sold under the name Gafquat by ISP, such as, for example, Gafquat 734 or Gafquat 755, or alternatively the products known as Copolymer 845, 958 and 937. These polymers are described in detail in French patents 2 077 143 and 2 393 573,
dimethylaminoethyl methacrylate/vinylcaprolactam/vinylpyrrolidone terpolymers, such as the product sold under the name Gaffix VC 713 by ISP,
vinylpyrrolidone/methacrylamidopropyldimethylamine copolymers, such as those sold under the name Styleze CC 10 by ISP,
quaternized vinylpyrrolidone/dimethylaminopropylmethacrylamide copolymers, such as the product sold under the name Gafquat HS 100 by ISP,
polymers, preferably crosslinked, of methacryloyloxy(C₁-C₄)alkyltri(C₁-C₄)alkylammonium salts, such as the polymers obtained by homopolymerization of dimethylaminoethyl methacrylate quaternized by methyl chloride or by copolymerization of acrylamide with dimethylaminoethyl methacrylate quaternized by methyl chloride, the homopolymerization or copolymerization being followed by a crosslinking by an olefinically unsaturated compound, in particular methylenebisacrylamide. Use may more particularly be made of a crosslinked acrylamide/methacryloyloxyethyltrimethylammonium chloride copolymer (20/80 by weight) in the form of a dispersion comprising 50% by weight of said copolymer in mineral oil. This dispersion is sold under the name Salcare® SC 92 by Ciba. Use may also be made of a crosslinked methacryloyloxyethyltrimethylammonium chloride homopolymer comprising approximately 50% by weight of the homopolymer in mineral oil or in a liquid ester. These dispersions are sold under the names Salcare® SC 95 and Salcare® SC 96 by Ciba.

(2) cationic polysaccharides, in particular cationic galactomannan gums and celluloses. Mention may more particularly be made, among the cationic polysaccharides, of cellulose ether derivatives comprising quaternary ammonium groups, cationic cellulose copolymers or cellulose derivatives grafted with a water-soluble quaternary ammonium monomer and cationic galactomannan gums.

The cellulose ether derivatives comprising quaternary ammonium groups are in particular described in FR 1 492 597, and mention may be made of the polymers sold under the name Ucare Polymer JR (JR 400 LT, JR 125 or JR 30M) or LR (LR 400 or LR 30M) by Amerchol. These polymers are also defined in the CTFA dictionary as quaternary ammoniums of hydroxyethylcellulose which has reacted with an epoxide substituted by a trimethylammonium group, such as, for example, polyquaternium-10.

The cationic cellulose copolymers or the cellulose derivatives grafted with a water-soluble quaternary ammonium monomer are described in particular in U.S. Pat. No. 4,131,576, and mention may be made of hydroxyalkyl celluloses, such as hydroxymethyl, hydroxyethyl or hydroxypropyl celluloses, grafted in particular with a methacryloyloxyethyltrimethylammonium, methacrylamidopropyltrimethylammonium or dimethyldiallylammonium salt, such as, for example, polyquaternium-4. The products marketed corresponding to this definition are more particularly the products sold under the names Celquat L 200 and Celquat H 100 by National Starch.

Use may also be made, among the cationic cellulose derivatives, of cationic associative celluloses, which can be chosen from quaternized cellulose derivatives, and in particular quaternized celluloses modified by groups comprising at least one fatty chain, such as linear or branched alkyl groups, linear or branched arylalkyl groups or linear or branched alkylaryl groups, preferably linear or branched alkyl groups, these groups comprising at least 8 carbon atoms, in particular from 8 to 30 carbon atoms, better still from 10 to 24, indeed even from 10 to 14, carbon atoms; or mixtures of these.

Preferably, mention may be made of quaternized hydroxyethylcelluloses modified by groups comprising at least one fatty chain, such as linear or branched alkyl groups, linear or branched arylalkyl groups or linear or branched alkylaryl groups, preferably linear or branched alkyl groups, these groups comprising at least 8 carbon atoms, in particular from 8 to 30 carbon atoms, better still from 10 to 24, indeed even from 10 to 14, carbon atoms; or mixtures of these.

Preferentially, mention may be made of the hydroxyethylcelluloses of formula (Ib):

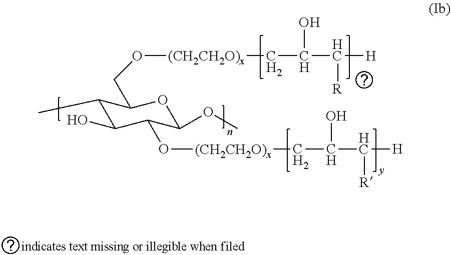

in which:
   R represents an ammonium group RaRbRcN⁺-Q⁻ in which Ra, Rb and Rc, which are identical or different, represent a hydrogen atom or a linear or branched C₁ to C₃₀ alkyl, preferably an alkyl, and Q⁻ represents an anionic counterion, such as a halide, for example a chloride or bromide;
   R′ represents an ammonium group R′aR′bR′cN⁺-Q′⁻ in which R′a, R′b and R′c, which are identical or different, represent a hydrogen atom or a linear or branched C₁ to C₃₀ alkyl, preferably an alkyl, and Q′⁻ represents an anionic counterion, such as a halide, for example a chloride or bromide;
it being understood that at least one of the radicals Ra, Rb, Rc, R′a, R′b and R′c represents a linear or branched C₈ to C₃₀ alkyl;
n, x and y, which are identical or different, represent an integer of between 1 and 10 000.

Preferably, in the formula (Ib), at least one of the radicals Ra, Rb, Rc, R′a, R′b or R′c represents a linear or branched C₈ to C₃₀, better still C₁₀ to C₂₄, indeed even C₁₀ to C₁₄, alkyl; mention may in particular be made of the dodecyl (C₁₂) radical.

Preferably, the other radical(s) represent a linear or branched C₁ to C₄ alkyl, in particular methyl.

Preferably, in the formula (Ib), only one of the radicals Ra, Rb, Rc, R′a, R′b or R′c represents a linear or branched C₈ to C₃₀, better still C₁₀ to C₂₄, indeed even C₁₀ to C₁₄, alkyl; mention may in particular be made of the dodecyl (C₁₂) radical. Preferably, the other radicals represent a linear or branched C₁ to C₄ alkyl, in particular methyl.

Better still, R can be a group chosen from —N⁺(CH₃)₃ Q′⁻ and

—N⁺(C₁₂H₂₅)(CH₃)₂ Q′⁻, preferably an —N⁺(CH₃)₃ Q′⁻ group.

Even better still, R′ can be an —N⁺(C₁₂H₂₅)(CH₃)₂ Q′⁻ group.

The aryl radicals preferably denote phenyl, benzyl, naphthyl or anthryl groups.

Mention may in particular be made of the polymers with the INCI names:
Polyquaternium-24, such as the product Quatrisoft LM 200*, sold by Amerchol/Dow Chemical;
PG-Hydroxyethylcellulose Cocodimonium Chloride, such as the product Crodacel QM®;
PG-Hydroxyethylcellulose Lauryldimonium Chloride (C₁₂ alkyl), such as the product Crodacel QL®; and
PG-Hydroxyethylcellulose Stearyldimonium Chloride (C₁₈ alkyl), such as the product Crodacel QS®, sold by Croda.

Mention may also be made of the hydroxyethylcelluloses of formula (Ib) in which R represents trimethylammonium halide and R′ represents dimethyldodecylammonium halide; preferentially, R represents trimethylammonium chloride (CH₃)₃N⁺— Cl⁻ and R′ represents dimethyldodecylammonium chloride (CH₃)₂(C₁₂H₂₅)N⁺— Cl⁻. This type of polymer is known under the INCI name Polyquaternium-67; mention may be made, as commercial products, of the Softcat Polymer SL® polymers, such as SL-100, SL-60, SL-30 and SL-5, from Amerchol/Dow Chemical.

More particularly, the polymers of formula (Ib) are, for example, those for which the viscosity is of between 2000 and 3000 cPs inclusive, preferentially between 2700 and 2800 cPs inclusive. Typically, Softcat Polymer SL-5 has a viscosity of 2500 cPs, Softcat Polymer SL-30 has a viscosity of 2700 cPs, Softcat Polymer SL-60 has a viscosity of 2700 cPs and Softcat Polymer SL-100 has a viscosity of 2800 cPs. Use may also be made of Softcat Polymer SX-1300X with a viscosity of between 1000 and 2000 cPs.

The cationic galactomannan gums are described more particularly in U.S. Pat. Nos. 3,589,578 and 4,031,307, and mention may be made of guar gums comprising cationic trialkylammonium groups. Use is made, for example, of guar gums modified by a 2,3-epoxypropyltrimethylammonium salt (for example a chloride). Such products are sold in particular under the names Jaguar C13 S, Jaguar C 15, Jaguar C 17, Jaguar C162 or Jaguar Excel by Rhodia. Such compounds have the INCI names guar hydroxypropyltrimonium chloride and hydroxypropyl guar hydroxypropyltrimonium chloride.

(3) polymers constituted of piperazinyl units and of divalent alkylene or hydroxyalkylene radicals having linear or branched chains, optionally interrupted by oxygen, sulfur or nitrogen atoms or by aromatic or heterocyclic rings, and also the oxidation and/or quaternization products of these polymers.

(4) water-soluble polyaminoamides, prepared in particular by polycondensation of an acid compound with a polyamine; these polyaminoamides can be crosslinked by an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively by an oligomer resulting from the reaction of a difunctional compound which is reactive with regard to a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyaminoamide; these polyaminoamides can be alkylated or, if they comprise one or more tertiary amine functional groups, quaternized.

(5) polyaminoamide derivatives resulting from the condensation of polyalkylenepolyamines with polycarboxylic acids followed by alkylation by difunctional agents. Mention may be made, for example, of adipic acid/dialkylaminohydroxyalkyldialkylenetriamine polymers in which the alkyl radical comprises from 1 to 4 carbon atoms and preferably denotes methyl, ethyl or propyl. Mention may more particularly be made, among these derivatives, of the adipic acid/dimethylaminohydroxypropyldiethylenetriamine polymers sold under the name Cartaretine F, F4 or F8 by Sandoz.

(6) polymers obtained by reaction of a polyalkylenepolyamine comprising two primary amine groups and at least one secondary amine group with a dicarboxylic acid chosen from diglycolic acid and saturated aliphatic dicarboxylic acids having from 3 to 8 carbon atoms; the molar ratio of the polyalkylenepolyamine to the dicarboxylic acid preferably being of between 0.8:1 and 1.4:1; the polyaminoamide resulting therefrom being reacted with epichlorohydrin in a molar ratio of epichlorohydrin, with respect to the secondary amine group of the polyaminoamide, preferably of between 0.5:1 and 1.8:1. Polymers of this type are sold in particular under the name Hercosett 57 by Hercules Inc. or else under the name PD 170 or Delsette 101 by Hercules in the case of the adipic acid/epoxypropyl/diethylenetriamine copolymer.

(7) alkyldiallylamine or dialkyldiallylammonium cyclopolymers, such as the homopolymers or copolymers comprising, as main constituent of the chain, units corresponding to the formula (VI) or (VII):

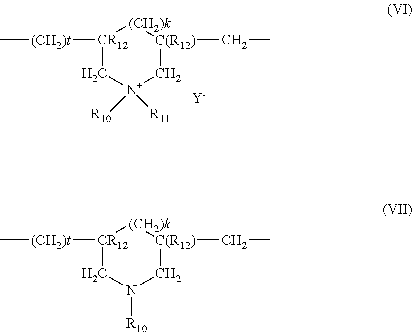

in which formulae (VI) and (VII):
   k and t are equal to 0 or 1, the sum k+t being equal to 1;
   R₁₂ denotes a hydrogen atom or a methyl radical;
   R₁₀ and R₁₁ denote, independently of each other, an alkyl group having from 1 to 6 carbon atoms, a hydroxyalkyl group in which the alkyl group has from 1 to 5 carbon atoms, a C₁ to C₄ amidoalkyl group; or alternatively R₁₀ and R₁₁ can denote, together with the nitrogen atom to which they are attached, heterocyclic groups, such as piperidinyl or morpholinyl; R₁₀ and R₁₁, independently of each other, preferably denote an alkyl group having from 1 to 4 carbon atoms; and
   Y⁻ is an anion, such as bromide, chloride, acetate, borate, citrate, tartrate, bisulfate, bisulfite, sulfate or phosphate.

Mention may more particularly be made of the dimethyldiallylammonium salt (for example chloride) homopolymer sold under the name Merquat 100 by Nalco (and its homologues of low weight-average molar masses) and the copolymers of diallyldimethylammonium salts (for example chloride) and of acrylamide, which are sold in particular under the names Merquat 550 and Merquat 7SPR.

(8) diquaternary ammonium polymers comprising repeat units of formula:

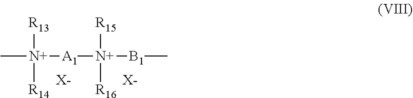

in which formula (VIII):
   R₁₃, R₁₄, R₁₅ and R₁₆, which are identical or different, represent aliphatic, alicyclic or arylaliphatic radicals comprising from 1 to 20 carbon atoms or lower hydroxyalkyl aliphatic radicals, or else R₁₃, R₁₄, R₁₅ and R₁₆, together or separately, constitute, with the nitrogen atoms to which they are attached, heterocycles optionally comprising a second heteroatom other than nitrogen, or else R₁₃, R₁₄, R₁₅ and R₁₆ represent a linear or branched C₁ to C₆ alkyl radical substituted by a nitrile, ester, acyl, amide or —CO—O—R₁₇-D or —CO—NH—R₁₇-D group, where R₁₇ is an alkylene and D is a quaternary ammonium group;
   A₁ and B₁ represent divalent polymethylene groups comprising from 2 to 20 carbon atoms which can be linear or branched and saturated or unsaturated and which can contain, bonded to or inserted in the main chain, one or more aromatic rings, or one or more oxygen or sulfur atoms or sulfoxide, sulfone, disulfide, amino, alkylamino, hydroxyl, quaternary ammonium, ureido, amide or ester groups, and
   X⁻ denotes an anion derived from an inorganic or organic acid;
it being understood that A₁, R₁₃ and R₁₅ can form, with the two nitrogen atoms to which they are attached, a piperazine ring;
in addition, if A₁ denotes a saturated or unsaturated and linear or branched alkylene or hydroxyalkylene radical, B₁ can also denote a (CH₂)ₙ—CO-D-OC—(CH₂)ₙ— group in which D denotes:
   a) a glycol residue of formula —O—Z—O—, where Z denotes a linear or branched hydrocarbon radical or a group corresponding to one of the following formulae: —(CH₂—CH₂—O)ₓ—CH₂—CH₂— and —[CH₂—CH(CH₃)—O]_{y}—CH₂—CH(CH₃)—, where x and y denote an integer from 1 to 4, representing a defined and unique degree of polymerization, or any number from 1 to 4, representing an average degree of polymerization;
   b) a bis-secondary diamine residue, such as a piperazine derivative;
   c) a bis-primary diamine residue of formula: —NH—Y—NH—, where Y denotes a linear or branched hydrocarbon radical, or alternatively the divalent radical —CH₂—CH₂—S—S—CH₂—CH₂—; or
   d) a ureylene group of formula: —NH—CO—NH—.

Preferably, X⁻ is an anion, such as chloride or bromide. These polymers have a number-average molar mass (Mn) generally of between 1000 and 100 000.

Mention may more particularly be made of the polymers which are constituted of repeat units corresponding to the formula:

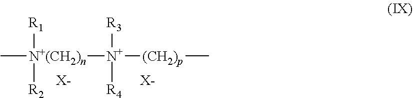

in which formula (IX) R₁, R₂, R₃ and R₄, which are identical or different, denote an alkyl or hydroxyalkyl radical having from 1 to 4 carbon atoms approximately, n and p are integers varying from 2 to 20 approximately and X⁻ is an anion derived from an inorganic or organic acid.

A compound of formula (IX) which is particularly preferred is that for which R₁, R₂, R₃ and R₄ represent a methyl radical, n=3, p=6 and X=Cl, which is known as Hexadimethrine chloride according to the INCI (CTFA) nomenclature.

(9) polyquaternary ammonium polymers comprising units of formula (X):

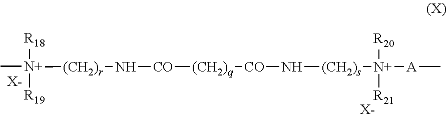

in which formula (X):
   R₁₈, R₁₉, R₂₀ and R₂₁, which are identical or different, represent a hydrogen atom or a methyl, ethyl, propyl, O-hydroxyethyl, β-hydroxypropyl or —CH₂CH₂(OCH₂CH₂)ₚOH radical, where p is equal to 0 or to an integer of between 1 and 6, with the proviso that R₁₈, R₁₉, R₂₀ and R₂₁ do not simultaneously represent a hydrogen atom,
   r and s, which are identical or different, are integers of between 1 and 6,
   q is equal to 0 or to an integer of between 1 and 34,
   X⁻ denotes an anion, such as a halide, and
   A denotes a radical of a dihalide or preferably represents —CH₂—CH₂—O—CH₂—CH₂—.

Mention may be made, for example, of the products Mirapol® A 15, Mirapol® AD1, Mirapol® AZ1 and Mirapol® 175 sold by Miranol.

(10) quaternary polymers of vinylpyrrolidone and of vinylimidazole, such as, for example, the products sold under the names Luviquat® FC 905, FC 550 and FC 370 by BASF.

(11) polyamines such as Polyquart® H sold by Cognis, referred to under the name Polyethylene Glycol (15) Tallow Polyamine in the CTFA dictionary.

(12) polymers comprising in their structure:
(a) one or more units corresponding to the following formula (A):

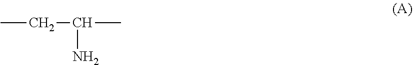

(b) optionally one or more units corresponding to the following formula (B):

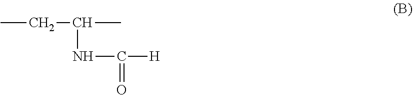

In other words, these polymers can in particular be chosen from homo- or copolymers comprising one or more units resulting from vinylamine and optionally one or more units resulting from vinylformamide.

(13) and their mixtures.

Preferably, these cationic polymers are chosen from polymers comprising, in their structure, from 5 mol % to 100 mol % of units corresponding to the formula (A) and from 0 mol % to 95 mol % of units corresponding to the formula (B), preferentially from 10 mol % to 100 mol % of units corresponding to the formula (A) and from 0 mol % to 90 mol % of units corresponding to the formula (B).

These polymers can be obtained, for example, by partial hydrolysis of polyvinylformamide. This hydrolysis can be carried out in acidic or basic medium.

The weight-average molecular weight of said polymer, measured by light scattering, can preferably vary from 1000 to 3 000 000 g/mol, more preferentially from 10 000 to 1 000 000 and more particularly still from 100 000 to 500 000 g/mol.

The cationic charge density of these polymers can preferably vary from 2 meq/g to 20 meq/g, more preferentially from 2.5 to 15 meq/g and more particularly from 3.5 to 10 meq/g.

The polymers comprising units of formula (A) and optionally units of formula (B) are sold in particular under the name Lupamin by BASF, such as, for example, and in a non-limiting way, the products provided under the names Lupamin 9095, Lupamin 5095, Lupamin 1095, Lupamin 9030 (or Luviquat 9030) and Lupamin 9010.

Preferably, the cationic polymer(s) are chosen from cationic polysaccharides, in particular associative or non-associative cationic polysaccharides, more preferentially non-associative cationic polysaccharides.

More preferentially, the cationic polymer(s) are chosen from cellulose ether derivatives comprising quaternary ammonium groups, cationic cellulose copolymers, cellulose derivatives grafted with a water-soluble quaternary ammonium monomer, cationic galactomannan gums and their mixtures.

More preferentially still, the cationic polymer(s) are chosen from cationic galactomannan gums, quaternary ammonium polymers of hydroxyethylcellulose which has reacted with an epoxide substituted by trimethylammonium groups, and their mixtures.

Very particularly preferably, the cationic polymer(s) are chosen from cationic guar gums, polyquaternium-10 and their mixtures.

Preferably, when the cationic polymer(s) are present in the anhydrous solid composition according to the invention, the total content of cationic polymer(s) ranges from 0.1% to 20% by weight, more preferentially from 0.5% to 15% by weight, more preferentially still from 1% to 10% by weight, even better still from 2% to 5% by weight, with respect to the total weight of the anhydrous solid composition.

Preferably, when the cationic polysaccharide(s) are present in the anhydrous solid composition according to the invention, the total content of cationic polysaccharide(s) ranges from 0.1% to 20% by weight, more preferentially from 0.5% to 15% by weight, more preferentially still from 1% to 10% by weight, even better still from 2% to 5% by weight, with respect to the total weight of the anhydrous solid composition.

Preferably, when the cationic galactomannan gum(s) and/or the quaternary ammonium polymer(s) of hydroxyethylcellulose which has reacted with an epoxide substituted by a trimethylammonium group are present in the anhydrous solid composition according to the invention, the total content of cationic galactomannan gum(s) and/or of quaternary ammonium polymer(s) of hydroxyethylcellulose which has reacted with an epoxide substituted by a trimethylammonium group ranges from 0.1% to 20% by weight, more preferentially from 0.5% to 15% by weight, more preferentially still from 1% to 10% by weight, even better still from 2% to 5% by weight, with respect to the total weight of the anhydrous solid composition.

The Non-Ionic Surfactants

The anhydrous solid composition according to the present invention may optionally further comprise one or more non-ionic surfactants.

The non-ionic surfactants which can be used according to the invention can be chosen from:
alcohols, α-diols and (C₁-C₂₀)alkylphenols, these compounds being polyethoxylated and/or polypropoxylated and/or polyglycerolated, it being possible for the number of ethylene oxide and/or propylene oxide groups to range from 1 to 100, and it being possible for the number of glycerol groups to range from 2 to 30; and/or these compounds comprising at least one fatty chain comprising from 8 to 40 carbon atoms, in particular from 10 to 20 carbon atoms; especially alcohols comprising at least one saturated or unsaturated and linear or branched C₈ to C₄₀ alkyl chain which are oxyethylenated and/or oxypropylenated, comprising from 1 to 100 mol of ethylene oxide and/or of propylene oxide, preferably from 2 to 50, more particularly from 2 to 40, mol of ethylene oxide and/or of propylene oxide;
condensates of ethylene oxide and of propylene oxide with fatty alcohols;
polyethoxylated fatty amides preferably having from 2 to 30 ethylene oxide units, polyglycerolated fatty amides comprising on average from 1 to 5 glycerol groups and in particular from 1.5 to 4;
ethoxylated fatty acid esters of sorbitan, preferably having from 2 to 40 ethylene oxide units;
fatty acid esters of sucrose;
polyoxyalkylenated, preferably polyoxyethylenated, fatty acid esters having from 2 to 150 mol of ethylene oxide, including oxyethylenated vegetable oils;
N—(C₆-C₂₄ alkyl)glucamine derivatives;
amine oxides, such as (C₁₀-C₁₄ alkyl)amine oxides or N—(C₁₀-C₁₄ acyl)aminopropylmorpholine oxides;
and their mixtures.

Mention may also be made of non-ionic surfactants of alkyl(poly)glycoside type, represented in particular by the following general formula:

R₁O—(R₂O)ₜ-(G)ᵥ
in which:
   R₁ represents a linear or branched alkyl or alkenyl radical comprising from 6 to 24 carbon atoms, in particular from 8 to 18 carbon atoms, or an alkylphenyl radical, the linear or branched alkyl radical of which comprises from 6 to 24 carbon atoms, in particular from 8 to 18 carbon atoms,
   R₂ represents an alkylene radical comprising 2 to 4 carbon atoms,
   G represents a sugar unit comprising from 5 to 6 carbon atoms,
   t denotes a value ranging from 0 to 10, preferably from 0 to 4,
   v denotes a value ranging from 1 to 15, preferably from 1 to 4.

Preferably, the alkyl(poly)glycoside surfactants are compounds of the formula described above in which:
R₁ denotes a saturated or unsaturated and linear or branched alkyl radical comprising from 8 to 18 carbon atoms,
R₂ represents an alkylene radical comprising from 2 to 4 carbon atoms,
t denotes a value ranging from 0 to 3, preferably equal to 0,
G denotes glucose, fructose or galactose, preferably glucose,
the degree of polymerization, that is to say the value of v, being able to range from 1 to 15, preferably from 1 to 4; the mean degree of polymerization more particularly being of between 1 and 2.

The glucoside bonds between the sugar units are generally of 1-6 or 1-4 type, preferably of 1-4 type. Preferably, the alkyl(poly)glycoside surfactant is an alkyl(poly)glucoside surfactant. Preference is very particularly given to C₈/C₁₆ alkyl (1,4)-(poly)glucosides, and in particular decyl glucosides and caprylyl/capryl glucosides.

Mention may be made, among the commercial products, of the products sold by Cognis under the Plantaren® (600 CS/U, 1200 and 2000) or Plantacare® (818, 1200 and 2000) names; the products sold by SEPPIC under the names Oramix CG 110 and Oramix® NS 10; the product sold by BASF under the name Lutensol GD 70, or the product sold by Chem Y under the name AG10 LK.

Preferably, use is made of C₈/C₁₆ alkyl (1,4)-(poly)glycosides, in particular as a 53% aqueous solution, such as those sold by Cognis under the reference Plantacare® 818 UP.

Preferentially, the non-ionic surfactant(s) are chosen from oxyalkylenated fatty alcohols comprising at least one saturated or unsaturated and linear or branched C₈ to C₄₀ alkyl chain and comprising a number of ethylene oxide and/or propylene oxide groups ranging from 1 to 100; more preferentially chosen from oxyethylenated and oxypropylenated fatty alcohols comprising at least one saturated or unsaturated and linear or branched C₈ to C₂₀, better still C₁₀ to C₁₈, alkyl chain and comprising a number of ethylene oxide and propylene oxide groups ranging from 2 to 50, better still from 2 to 40, such as PPG-5-Ceteth-20.

Preferably, when the non-ionic surfactant(s) are present in the anhydrous solid composition according to the invention, the total content of non-ionic surfactant(s) ranges from 0.1% to 15% by weight, more preferentially from 0.5% to 10% by weight, more preferentially still from 1% to 5% by weight, with respect to the total weight of the anhydrous solid composition.

Preferably, when the oxyalkylenated fatty alcohol(s) are present in the anhydrous solid composition according to the invention, the total content of oxyalkylenated fatty alcohol(s) ranges from 0.1% to 15% by weight, more preferentially from 0.5% to 10% by weight, more preferentially still from 1% to 5% by weight, with respect to the total weight of the anhydrous solid composition.

The anhydrous solid composition can optionally additionally comprise one or more silicones, preferably chosen from aminosilicones, in particular in a content ranging from 0.01% to 10% by weight, more preferentially from 0.1% to 7% by weight, more preferentially still from 0.5% to 5% by weight, with respect to the total weight of the anhydrous solid composition.

The term “silicone” is understood to mean any organosilicon polymer or oligomer having a linear or cyclic, branched or crosslinked, structure, of variable molecular weight, obtained by polymerization and/or by polycondensation of suitably functionalized silanes and essentially constituted by a repetition of main units in which the silicon atoms are connected to one another by oxygen atoms (siloxane —Si—O—Si— bond), optionally substituted hydrocarbon radicals being directly connected via a carbon atom to said silicon atoms; and more particularly dialkylsiloxane polymers, aminated silicones or dimethiconols.

The term “aminosilicone” denotes any silicone comprising at least one primary, secondary or tertiary amine or one quaternary ammonium group.

The anhydrous solid composition according to the invention can additionally contain additives normally used in cosmetics, such as preservatives, fragrances and colorants.

These additives, preferably in the form of powders, can be present in the composition according to the invention in an amount ranging from 0% to 20% by weight, with respect to the total weight of the composition.

A person skilled in the art will take care to choose these optional additives and their amounts so that they do not harm the properties of the anhydrous solid compositions of the present invention.

According to a preferred embodiment of the invention, the anhydrous solid composition comprises:
(i) one or more sulfate anionic surfactants,
(ii) one or more amphoteric or zwitterionic surfactants chosen from (C₈-C₂₀)alkyl betaine(s), (C₈-C₂₀)alkylamido(C₃-C₈)alkyl betaine(s) and their mixtures, and
(iii) from 0.1% to 15% by weight, with respect to the total weight of the anhydrous solid composition, of one or more C₁₀-C₂₀ fatty acid salts, and
the total content of surfactants being greater than or equal to 60% by weight, with respect to the total weight of the anhydrous solid composition.

According to a particularly preferred embodiment of the invention, the anhydrous solid composition comprises:
(i) one or more sulfate anionic surfactants,
(ii) one or more amphoteric or zwitterionic surfactants chosen from (C₈-C₂₀)alkyl betaine(s), (C₈-C₂₀)alkylamido(C₃-C₈)alkyl betaine(s) and their mixtures.
(iii) from 0.1% to 15% by weight, with respect to the total weight of the anhydrous solid composition, of one or more C₁₀-C₂₀, better still C₁₂-C₁₈, fatty acid salts, more preferentially still magnesium stearate,
(iv) optionally one or more cationic polymers, preferably chosen from cationic polysaccharides, more preferentially chosen from cationic guar gums, polyquarternium-10 and their mixtures, and
(v) optionally one or more non-ionic surfactants, preferably one or more oxyalkylenated C₈-C₄₀ fatty alcohols,
the total content of surfactants being greater than or equal to 60% by weight, preferably ranging from 60% to 95% by weight, better still from 65% to 90% by weight and even better still from 70% to 85% by weight, with respect to the total weight of the anhydrous solid composition.

The Preparation Method

Another subject-matter of the invention is a method for the preparation of an aqueous composition for washing, and optionally in addition for conditioning, keratin fibres, in particular human keratin fibres, such as the hair, comprising at least a step of mixing an anhydrous solid composition as described above with water.

The mixing can be carried out by hand, for example by shaking and/or inverting one or more times the container containing the anhydrous solid composition and water.

Preferably, the mixing step lasts between 1 and 120 seconds, more preferentially between 2 and 60 seconds, more preferentially still between 3 and 30 seconds and better still between 5 and 20 seconds.

Advantageously, once the mixing step has been carried out, the anhydrous solid composition may be completely dissolved. However, it is preferable to leave standing the aqueous composition resulting from the mixing.

According to the method of the invention, said mixing step is preferably followed by a step consisting in leaving the aqueous composition resulting from said mixing standing for at least 20 minutes at ambient temperature (25° C.) and atmospheric pressure, more preferentially at least 25 minutes, more preferentially still 30 minutes, better still 60 minutes, even better still 90 minutes, still better 2 hours, still better 4 hours, still better 6 hours, still better 8 hours, still better 10 hours, indeed even 12 hours, indeed even again 18 hours, and very particularly preferably at least 24 hours, before it is used on keratin fibres.

The resting time makes it possible in particular to eliminate bubbles from the composition and can thus result in a more transparent or translucent composition when it is left at rest for a longer time.

Preferably, during the mixing step of the method according to the invention, the ratio by weight of the total content of anhydrous solid composition, on the one hand, to the total content of water, on the other hand, ranges from 0.01 to 0.5, more preferentially from 0.05 to 0.4 and more preferentially still from 0.1 to 0.3.

The aqueous composition obtained by the preparation method according to the invention is preferably a shampoo.

Preferably, the aqueous composition obtained by the preparation method according to the invention is transparent.

It has been noticed that the transparency of the aqueous composition obtained is particularly attractive and desired by users, in particular when the aqueous composition obtained is packaged in a transparent container.

The term “transparent” composition is understood to mean a composition through which it is possible to see clearly with the naked eye.

The transparency of the composition can be characterized by measuring its transmittance.

In the context of the present invention, the transmittance measurements were carried out at 25° C. and at atmospheric pressure, with a Cary Type 100 scan UV-visible spectrophotometer.

Preferably, the transmittance of the composition according to the invention, measured at ambient temperature (25° C.) and atmospheric pressure, is greater than or equal to 80%, preferentially greater than or equal to 85%, more preferentially still greater than or equal to 90%, even better still greater than or equal to 92%; and in particular ranging from 80% to 100%, indeed even from 85% to 100%, in particular from 90% to 100%, indeed even from 92% to 100%.

Preferably, the pH of said aqueous composition resulting from said mixing is of between 3 and 8, more preferentially between 3.5 and 7 and more preferentially still between 4.5 and 5.5.

Another subject-matter of the invention is the use of an anhydrous solid composition as described above for washing, and optionally in addition for conditioning, keratin fibres, in particular human keratin fibres, such as the hair.

The invention also relates to a method for the treatment of keratin fibres, comprising the application, to said keratin fibres, of the anhydrous solid composition as described above.

Preferably, the method for the treatment of keratin fibres is a method for washing, and optionally in addition for conditioning, keratin fibres, such as hair.

The invention also relates to a kit for the preparation of an aqueous composition for washing, and optionally in addition for conditioning, keratin fibres, in particular human keratin fibres, such as the hair, comprising at least a first compartment comprising an anhydrous solid composition as described above and at least a second compartment.

Said second compartment is capable of containing the anhydrous solid composition as described above and the amount of water necessary to carry out the mixing according to the method of the invention, for example a container, such as a glass bottle.

Preferably, the second compartment is transparent.

The examples which follow serve to illustrate the invention without, however, exhibiting a limiting nature.

### EXAMPLES

### Example 1

The anhydrous solid compositions A to E according to the invention are prepared from the ingredients shown in the tables below, the amounts of which are expressed as % by weight of active material (AM).

TABLE 1

| | Composition A | Composition B |
|---|---|---|
| Sodium lauryl sulfate | 42.1 | 59.2 |
| Sodium coco sulfate | 20.8 | — |
| Cocamidopropyl betaine | 16.2 | 20.9 |
| Hydroxypropyl guar | 3.3 | — |
| hydroxypropyltrimonium | | |
| chloride | | |
| Polyquaternium-10 | — | 3.1 |
| PPG-5-Ceteth-20 | 2.5 | 2.6 |
| Magnesium stearate | 3 | 1 |
| Preservatives | 5.9 | 4.8 |
| Ethyl lactate | — | 1 |
| Sodium chloride | 2.9 | 3.7 |
| Fragrance | 3.3 | 3.7 |

TABLE 2

| | Composition C | Composition D |
|---|---|---|
| Sodium lauryl sulfate | 70.5 | 68.9 |
| Cocamidopropyl betaine | 9.1 | 8.9 |
| Hydroxypropyl guar | 3.1 | 4.8 |
| hydroxypropyltrimonium | | |
| chloride | | |
| Guar hydroxypropyltrimonium | 1.8 | — |
| chloride | | |
| Amodimethicone | — | 3 |
| Magnesium stearate | 3 | 3 |
| Preservatives | 7.2 | 6.3 |
| Sodium chloride | 1.6 | 1.6 |
| Fragrance | 3.7 | 3.5 |

TABLE 3

| | Composition E |
|---|---|
| Sodium lauryl sulfate | 65.8 |
| Cocamidopropyl betaine | 14.3 |
| Hydroxypropyl guar | 1.7 |
| hydroxypropyltrimonium | |
| chloride | |
| Polyquaternium-10 | 1.7 |
| Magnesium stearate | 5 |
| Preservatives | 6.9 |
| Sodium chloride | 1.2 |
| Fragrance | 3.4 |

Protocol:

Each anhydrous solid composition (A to E) according to the invention was poured into a separate transparent glass bottle comprising water, according to the respective amounts shown in Table 4 below.

TABLE 4

| Solid compositions | A | B | C | D | E |
|---|---|---|---|---|---|
| Amount of anhydrous | 45.7 | 48.6 | 40.8 | 41.7 | 43.7 |
| solid composition added | | | | | |
| (in g) | | | | | |
| Amount of water added | 204.3 | 201.4 | 209.2 | 208.3 | 206.3 |
| (in g) | | | | | |
| Ratio by weight of the | 0.22 | 0.24 | 0.20 | 0.20 | 0.21 |
| amount of anhydrous | | | | | |
| solid composition to the | | | | | |
| amount of water | | | | | |

Then the five closed bottles were shaken and inverted several times by hand, in order to respectively obtain the aqueous compositions M_{A} to M_{E}.

The aqueous compositions M_{A} to M_{E} were subsequently left standing at ambient temperature (25° C.) and at atmospheric pressure for 24 hours.

After standing for 24 hours, it is observed that the aqueous compositions M_{A} to M_{D} are transparent and that the aqueous composition M_{E} is opaque. In particular, it was observed that the aqueous composition M_{A} exhibits a transmittance of 97%.

After standing for 24 hours, the aqueous compositions M_{A} to M_{E} were applied to locks of hair (2 g of composition/g of lock of hair). The locks were subsequently left in the open air for a leave-on time of 5 min and finally rinsed with clear water.

It was observed that the aqueous compositions M_{A} to M_{E} exhibit a good washing power and provide the hair with good cosmetic properties. In particular, these aqueous compositions M_{A} to M_{E} contribute suppleness, a soft feel, sheen and ease of disentangling of the hair.

### Example 2

The anhydrous solid composition F according to the invention and the comparative anhydrous solid composition G are prepared from the ingredients indicated in the tables below, the amounts of which are expressed as weight percentages of active material (AM).

TABLE 5

| | Composition F | Composition G |
|---|---|---|
| | (Invention) | (Comparative) |
| Sodium lauryl sulfate | 61 | 61 |
| Cocamidopropylbetaine | 15.3 | 15.3 |
| Hydroxypropyl guar | 2.9 | 2.9 |
| hydroxypropyltrimonium chloride | | |
| Sodium benzoate | 3.6 | 3.6 |
| Citric acid | 4.6 | 4.6 |
| Magnesium stearate | 5 | — |
| Magnesium carbonate | — | 5 |
| Sodium chloride | 3.3 | 3.3 |
| Fragrance | 4.3 | 4.3 |

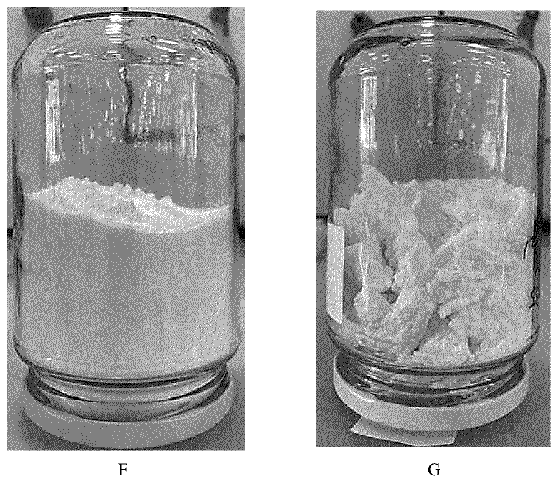

FIG. 1 shows the photographs of the anhydrous solid compositions F and G before their mixtures with water. The photograph on the left represents the anhydrous solid composition F according to the invention. The photograph on the right represents the comparative anhydrous solid composition G.

As it can be observed in FIG. 1, composition F according to the invention has a smooth, fluid appearance, whereas comparative composition H is agglomerated, in the form of packets. The appearance of composition F according to the invention promotes mixing with water.

Protocol:

Each anhydrous solid composition F and G was poured into a different transparent glass bottle comprising water, in the following amounts: 13.9 g of composition+86.1 g of water.

The two closed bottles were then shaken and inverted several times by hand to obtain, respectively, the aqueous compositions M_{F} (invention) and M_{G} (comparative).

The aqueous compositions M_{F} and M_{G} were then left to stand for 12 hours at room temperature (25° C.) and at atmospheric pressure.

Evaluation and Results:

1/ The Ford cup viscosity (diameter 8 mm) of each composition is measured after preparation. This method consists in measuring, at a determined temperature, the flow time of 90 g of composition through an orifice of determined diameter. The result is expressed in seconds.

TABLE 6

| M_{F} | M_{G} |
|---|---|
| (invention) | (comparative) |
| 70 s | 121 s |

It is observed that the comparative aqueous composition M_{G}, obtained from the comparative anhydrous solid composition G, exhibits a very high viscosity, incompatible with good properties of use (it is difficult to get the composition out of the container), unlike the aqueous composition M_{F} according to the invention.

2/ The aqueous compositions M_{F} and M_{G} were applied to locks of moderately sensitized hair (alkaline solubility=20%, SA20) at a rate of 0.4 g of composition per gram of hair. The aqueous compositions M_{F} and M_{G} are left on the hair for 30 s, then rinsed with water.

The softness performances were evaluated on wet hair by an expert, during a blind test, assigning a:
“+” to the lock of hair with the best results
“−” to the lock of hair with the least good results.

To evaluate the softness, you grab the lock between your thumb and forefinger and slide your fingers along the lock from the roots to the tips.

TABLE 7

| | M_{F} | M_{G} |
|---|---|---|
| | (invention) | (comparative) |
| Softness | + | − |

The aqueous composition M_{F} according to the invention exhibits improved performance in terms of softness, compared to the comparative aqueous composition M_{G}.

1. Anhydrous solid composition comprising:
(i) one or more anionic surfactants,
(ii) one or more amphoteric or zwitterionic surfactants, and
(iii) from 0.1% to 15% by weight, with respect to the total weight of the anhydrous solid composition, of one or more C₈-C₃₂ fatty acid salts; and
the total content of surfactants being greater than or equal to 60% by weight, with respect to the total weight of the anhydrous solid composition.

2. Composition according to claim 1, characterized in that the anionic surfactant(s) are chosen from sulfate anionic surfactants;
preferably from alkyl sulfates, alkyl ether sulfates, alkylamido ether sulfates, alkylaryl polyether sulfates, monoglyceride sulfates; mixtures thereof and also the salts of these compounds;
more preferentially from C₆-C₃₀, better still C₈-C₂₄, even better still C₁₀-C₂₄, indeed even C₁₂-C₂₂, alkyl sulfates, C₆-C₂₄, better still C₁₀-C₂₄, indeed even C₁₂-C₂₂, alkyl ether sulfates, preferably comprising from 1 to 20 ethylene oxide units, and their mixtures;
more preferably still from C₆-C₃₀, better still C₈-C₂₄, even better still C₁₀-C₂₄, indeed even C₁₂-C₂₂, alkyl sulfates.

3. Composition according to either one of claim 1, characterized in that the total content of anionic surfactant(s) ranges from 55% to 90% by weight, preferably from 56% to 85% by weight, more preferentially from 57% to 80% by weight, better still from 58% to 75% by weight, even better still from 59% to 75% by weight and still better from 60% to 75% by weight, with respect to the total weight of the anhydrous solid composition.

4. Composition according to claim 1, characterized in that the amphoteric or zwitterionic surfactant(s) are chosen from (C₈-C₂₀)alkyl betaines, (C₈-C₂₀)alkylamido(C₃-C₈)alkyl betaines, (C₈-C₂₀)alkylamphoacetates, (C₈-C₂₀)alkylamphodiacetates and their mixtures, preferably from (C₈-C₂₀)alkyl betaines, (C₈-C₂₀)alkylamido(C₃-C₈)alkyl betaines and their mixtures.

5. Composition according to claim 1, characterized in that the total content of amphoteric or zwitterionic surfactant(s) ranges from 5% to 40% by weight, preferably from 6% to 35% by weight, more preferentially from 7% to 30% by weight and better still from 8% to 20% by weight, with respect to the total weight of the anhydrous solid composition.

6. Composition according to claim 1, characterized in that the total content of surfactants ranges from 60% to 95% by weight, preferably from 65% to 90% by weight and more preferentially from 70% to 85% by weight, with respect to the total weight of the anhydrous solid composition.

7. Composition according to claim 1, characterized in that the C₈-C₃₂ fatty acid salt(s) are chosen from C₁₀-C₂₀ fatty acid salts, preferably from C₁₂-C₁₈ fatty acid salts, more preferentially from alkali metal and alkaline earth metal salts of stearic acid, and their mixtures, and more preferentially still the C₈-C₃₂ fatty acid salt is magnesium stearate.

8. Composition according to claim 1, characterized in that the total content of C₈-C₃₂ fatty acid salt(s) ranges from 0.5% to 15% by weight, preferably from 0.5% to 10% by weight and more preferentially from 1% to 8% by weight, with respect to the total weight of the anhydrous solid composition.

9. Composition according to claim 1, in that it further comprises one or more cationic polymers preferably chosen from:
(1) homopolymers or copolymers derived from acrylic or methacrylic esters or amides and comprising at least one of the units of following formulae: 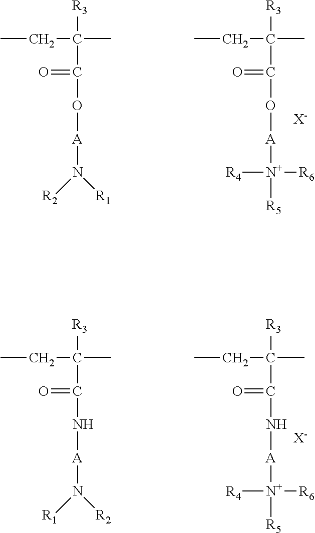
in which formulae:
R₃, which are identical or different, denote a hydrogen atom or a CH₃ radical;
A, which are identical or different, represent a linear or branched divalent alkyl group of 1 to 6 carbon atoms, preferably 2 or 3 carbon atoms, or a hydroxyalkyl group of 1 to 4 carbon atoms;
R₄, R₅ and R₆, which are identical or different, represent an alkyl group having from 1 to 18 carbon atoms or a benzyl radical, preferably an alkyl group having from 1 to 6 carbon atoms;
R₁ and R₂, which are identical or different, represent a hydrogen atom or an alkyl group having from 1 to 6 carbon atoms, preferably methyl or ethyl; and
X denotes an anion derived from an inorganic or organic acid, such as a methosulfate anion or a halide, such as chloride or bromide,
(2) cationic polysaccharides, such as cellulose ether derivatives comprising quaternary ammonium groups, cationic cellulose copolymers, cellulose derivatives grafted with a water-soluble quaternary ammonium monomer and cationic galactomannan gums,
(3) polymers constituted of piperazinyl units and of divalent alkylene or hydroxyalkylene radicals having linear or branched chains, optionally interrupted by oxygen, sulfur or nitrogen atoms or by aromatic or heterocyclic rings, and also the oxidation and/or quaternization products of these polymers,
(4) water-soluble polyaminoamides, prepared in particular by polycondensation of an acid compound with a polyamine,
(5) polyaminoamide derivatives resulting from the condensation of polyalkylenepolyamines with polycarboxylic acids followed by alkylation by difunctional agents,
(6) polymers obtained by reaction of a polyalkylenepolyamine comprising two primary amine groups and at least one secondary amine group with a dicarboxylic acid chosen from diglycolic acid and saturated aliphatic dicarboxylic acids having from 3 to 8 carbon atoms;
(7) alkyldiallylamine or dialkyldiallylammonium cyclopolymers,
(8) diquaternary ammonium polymers comprising repeat units of formula (VIII): 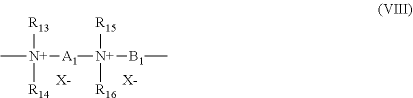
in which formula (VIII):
R₁₃, R₁₄, R₁₅ and R₁₆, which are identical or different, represent aliphatic, alicyclic or arylaliphatic radicals comprising from 1 to 20 carbon atoms or lower hydroxyalkyl aliphatic radicals, or else R₁₃, R₁₄, R₁₅ and R₁₆, together or separately, constitute, with the nitrogen atoms to which they are attached, heterocycles optionally comprising a second heteroatom other than nitrogen, or else R₁₃, R₁₄, R₁₅ and R₁₆ represent a linear or branched C₁ to C₆ alkyl radical substituted by a nitrile, ester, acyl, amide or —CO—O—R₁₇-D or —CO—NH—R₁₇-D group, where R₁₇ is an alkylene and D is a quaternary ammonium group;
A₁ and B₁ represent divalent polymethylene groups comprising from 2 to 20 carbon atoms which can be linear or branched and saturated or unsaturated and which can contain, bonded to or inserted in the main chain, one or more aromatic rings, or one or more oxygen or sulfur atoms or sulfoxide, sulfone, disulfide, amino, alkylamino, hydroxyl, quaternary ammonium, ureido, amide or ester groups, and
X⁻ denotes an anion derived from an inorganic or organic acid;
it being understood that A₁, R₁₃ and R₁₅ can form, with the two nitrogen atoms to which they are attached, a piperazine ring;
in addition, if A₁ denotes a saturated or unsaturated and linear or branched alkylene or hydroxyalkylene radical, B₁ can also denote a (CH₂)ₙ—CO-D-OC—(CH₂)ₙ— group in which D denotes:
a) a glycol residue of formula —O—Z—O—, where Z denotes a linear or branched hydrocarbon radical or a group corresponding to one of the following formulae: —(CH₂—CH₂—O)ₓ—CH₂—CH₂— and —[CH₂—CH(CH₃)—O]_{y}—CH₂—CH(CH₃)—, where x and y denote an integer from 1 to 4, representing a defined and unique degree of polymerization, or any number from 1 to 4, representing an average degree of polymerization;
b) a bis-secondary diamine residue, such as a piperazine derivative;
c) a bis-primary diamine residue of formula: —NH—Y—NH—, where Y denotes a linear or branched hydrocarbon radical, or alternatively the divalent radical —CH₂—CH₂—S—S—CH₂—CH₂—; or
d) a ureylene group of formula: —NH—CO—NH—,
(9) polyquaternary ammonium polymers comprising units of formula (X): 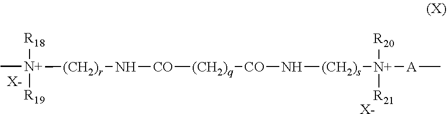
in which formula (X):
R₁₈, R₁₉, R₂₀ and R₂₁, which are identical or different, represent a hydrogen atom or a methyl, ethyl, propyl, β-hydroxyethyl, β-hydroxypropyl or —CH₂CH₂(OCH₂CH₂)ₚOH radical, where p is equal to 0 or to an integer of between 1 and 6, with the proviso that R₁₈, R₁₉, R₂₀ and R₂₁ do not simultaneously represent a hydrogen atom,
r and s, which are identical or different, are integers of between 1 and 6,
q is equal to 0 or to an integer of between 1 and 34,
X⁻ denotes an anion, such as a halide, and
A denotes a radical of a dihalide or preferably represents —CH₂—CH₂—O—CH₂—CH₂—;
(10) quaternary polymers of vinylpyrrolidone and of vinylimidazole,
(11) polyamines; and
(12) polymers comprising in their structure:
(a) one or more units corresponding to the following formula (A): 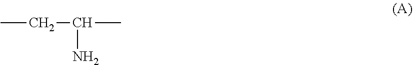
(b) optionally one or more units corresponding to the following formula (B): 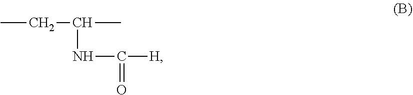
(13) and their mixtures;
more preferentially, the cationic polymer(s) are chosen from cationic polysaccharides.

10. Composition according to claim 1, characterized in that it further comprises at least one non-ionic surfactant, preferably chosen from oxyalkylenated fatty alcohols comprising at least one saturated or unsaturated and linear or branched C₈ to C₄₀ alkyl chain and comprising a number of ethylene oxide and/or propylene oxide groups ranging from 1 to 100; more preferentially chosen from oxyethylenated and oxypropylenated fatty alcohols comprising at least one saturated or unsaturated and linear or branched C₈ to C₂₀, better still C₁₀ to C₁₈, alkyl chain and comprising a number of ethylene oxide and propylene oxide groups ranging from 2 to 50, better still from 2 to 40.

11. Method for the preparation of an aqueous composition for washing, and optionally additionally conditioning, keratin fibres, in particular human keratin fibres, such as the hair, comprising at least a step of mixing an anhydrous solid composition as defined in claim 1 with water, optionally followed by a step consisting in leaving the aqueous composition resulting from said mixing standing for at least 20 minutes before it is used on keratin fibres, preferably at least 30 minutes, better still 60 minutes, even better still 90 minutes, still better 2 hours, indeed even 4 hours, and very particularly preferably at least 24 hours, before it is used on keratin fibres.

12. Method according to claim 11, characterized in that the ratio by weight of the total content of anhydrous solid composition, on the one hand, to the total content of water, on the other hand, ranges from 0.01 to 0.5, preferably from 0.05 to 0.4 and more preferentially from 0.1 to 0.3.

13. Use of an anhydrous solid composition as defined in claim 1 for the washing, and optionally additionally the conditioning, of keratin fibres, in particular human keratin fibres, such as the hair.

14. Method for the treatment of keratin fibres comprising the application, to said keratin fibres, of an anhydrous solid composition as defined in claim 1.

15. Kit for the preparation of an aqueous composition for washing, and optionally in addition for conditioning, keratin fibres, in particular human keratin fibres, such as the hair, comprising at least a first compartment comprising an anhydrous solid composition as defined in claim 1 and at least a second, preferably transparent, compartment capable of containing the mixture of said anhydrous solid composition with water.
